(19) United States
(12) Patent Application Publication
(10) Pub. No.: US 2009/0314639 A1
(43) Pub. Date: Dec. 24, 2009

(54) MEANS AND DEVICES FOR ELECTRO-FILTRATION OF MOLECULES

(76) Inventors: Michel Daniel Faupel, Echentzwiller (FR); Jan Van Oostrum, Fluh (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/441,948

(22) PCT Filed: Sep. 17, 2007
(86) PCT No.: PCT/EP2007/008074

§ 371 Date: Mar. 19, 2009

(30) Foreign Application Priority Data
Sep. 19, 2006 (EP) 06120904.5

(51) Int. Cl. B01D 57/02; C07C 237/06; C07C 233/05

(52) U.S. Cl. 204/459; 564/198; 564/160; 204/469; 204/606

(57) ABSTRACT

The present invention relates to means and devices for electro-filtration of molecules. The membranes comprise N-acryloyl-tris(hydroxymethyl)aminomethane (NAT) covalently linked to a support. The invention further encompasses compositions comprising an isoelectric buffer covalently bound to N-acryloyl-tris(hydroxymethyl)aminomethane (NAT). In particular, the present invention relates to membranes and devices allowing an isoelectric filtration of molecules in solution.

### SCOPE OF THE INVENTION

The present invention relates to means and devices for electro-filtration of molecules. In particular, the present invention relates to membranes and devices allowing an isoelectric filtration of molecules in solution.

### BACKGROUND

Preparative electrophoresis is a known technique and various forms of electrophoresis apparatus have been proposed for both analytical and preparative purposes. Basically, the instrumentations and principles for preparative electrophoresis can be classified into four main classes, namely disc electrophoresis, free curtain electrophoresis, isotachophoresis and isoelectric focusing (A. T. Andrews, Electrophoresis: Theory, Techniques, and Biochemical and Clinical Applications, Clarendon Press, Oxford 1986, and P. G. Righetti, Isoelectric Focusing: Theory, Methodology and Applications, Elsevier, Amsterdam, pp. 204-207 (1983)).

In general, disc electrophoresis and isotachophoresis are run in hydrophilic matrices, either continuous (agarose and polyacrylamide) or discontinuous (granulated beds, such as Sephadex.RTM.). They are characterized by a high resolving power, but only tolerate low sample loads. Free curtain electrophoresis in general utilizes continuous buffers, is performed in a free liquid phase and is characterized by a continuously flowing thin film of buffer with a continuous sample input. Basically, this technique offers large sample handling capacities but possesses a low resolution. In addition, due to the higher diffusion coefficient of proteins, this method is mostly confined to purification of intact cells or subcellular organelles.

Isoelectric focusing (IEF) can be performed either in liquid supports (density gradients) or in gel media, either continuous or granulated. Today, most IEF applications are performed in gelatinous supporting media (such as agarose and polyacrylamide matrices). The technique allows a high resolving power, but only tolerates moderate protein loads. In addition, all preparative techniques utilising as anticonvective media hydrophilic gels have the problem of recovering the purified protein from the matrix. This requires additional handling steps, e.g. detection of the zone of interest, band cutting and elution by diffusion or electrophoretic recovery. Those techniques have two major disadvantages: (a) low recoveries, as any matrix tends to irreversibly adsorb proteins, and (b) the possibility of contamination from gel material (especially in the case of synthetic supports, such as polyacrylamide, which is a toxic molecule, contamination from unreacted monomers, and from short oligomeric polyacrylamide coils which were non-covalently grafted to the bulk matrix). The use of membranes with a set pH value is used, e.g. in the technique described by Möller et al, Electrophoresis, 2005, 26, 35-46, wherein several membranes with fixed pH are used for separating biomolecules. The technique described therein is, however, time consuming, has relatively low protein load capacity and requires a cooling system to avoid over-heating. In addition, this technique is based on the use of acrylamide matrices.

The prior art discloses many types of devices comprising two subcompartments that are separated from each other by septa-like structure, for example, monofilament screens, membranes, gels, filters, and fritted discs. Generally, these devices are assembled from a plurality of essentially parallel frames, chambers, compartments or spacers, separated from each other by the septa. Multicompartment electrolyzers with isoelectric membranes were introduced (Righetti, P. G., Wenisch, E. and Faupel, M., 1989, J. Chromatogr. 475:293-309; Righetti, P. G., Wenisch, E., Jungbauer, A., Katinger, H. and Faupel. M., 1990, J. Chromatogr. 500:681-696; Righetti, P. G., Faupel, M. and Wenisch, E., 1992, In: Advances in Electrophoresis, Vol. 5, Chrambach, A., Dunn, M. J. and Radola, B. J., eds., VCH, Weinheim, pp. 159-200) for processing large volumes and amounts of proteins to homogeneity. This purification procedure, based on isoelectric focusing, progresses under recycling conditions, by keeping the protein macroions in a reservoir and continuously passing them in the electric field across a multicompartment electrolyzer equipped with zwitterionic membranes. In this system, the protein is always kept in a liquid vein (thus it is not lost by adsorption onto surfaces, as customary in chromatographic procedures) and it is trapped into a chamber delimited by two membranes having pIs encompassing the pI value of the protein being purified. Thus, by a continuous titration process, all other impurities, either non isoelectric or having different pI values, are forced to leave the chamber, in which the protein of interest will ultimately be present as the sole species, characterized by being isoelectric and isoionic as well (note that the isoelectric and isoionic points of a protein can differ to some extent in the presence of counterions).

In the original patent describing this process (Faupel, D. M. and Righetti, P. G., U.S. Pat. No. 4,971,670, Nov. 20, 1990) an isoelectric focusing process in the presence of buffering isoelectric membranes was developed solely and exclusively for separation and purification of proteins and peptides, i.e. for removing contaminants (including other macroions and salts) from the protein of interest. Isoelectric membranes have also been described by Martin, A. J. P. and Hampson, F. (U.S. Pat. No. 4,243,507, 1981), where they are used for suppressing electrosmotic flow generated by fixed or adsorbed charges on the electrophoretic cell. Righetti, P. G. (U.S. Pat. No. 5,834,272, Nov. 10, 1998) described a method for immobilizing enzymes, while still keeping them in solution, thus under conditions of homogeneous catalysis. This method consists in blocking enzymes in between two isoelectric membranes, having isoelectric points (pI) on either side of the pI of the enzyme to be “trapped”. The reactor consists on a multichamber electrolyzer, in which the electric field is coupled to a hydraulic flow for continuously recycling the enzyme inside and outside the electric field to reservoirs acting as both heat exchangers and as feeders for injecting (or collecting) substrates, cofactors and other reagents. The pH of optimum activity is maintained by co-immobilizing the buffers within the enzyme reaction chamber. This is achieved by selecting appropriate amphoteric buffers, having a pI value comprised between the pI of the two membranes keeping the enzyme isoelectric and possessing a reasonable buffering power at their respective pls. In U.S. Pat. No. 4,362,612 issued to Bier, the adjoining compartments are functionally designed to electrophoretically adjust to different pH values, thereby separating dissolved proteins according to their isoelectric points. Similar multiple subcompartments devices are described in U.S. Pat. No. 4,971,670 issued to Faupel et al., U.S. Pat. No. 5,173,164 issued to Egen et al., U.S. Pat. No. 4,963,236 issued to Rodkey et al., and U.S. Pat. No. 5,087,338 issued to Perry et al., all of which disclose devices comprising series of parallel spacers that are separated from each other by septa, which results in an essentially parallel array of subcompartments. A large number of other similar devices have been disclosed in patents and other publications. In all such devices, electrodes are provided at the ends of the assembly of subcompartments for the application of an electrical field.

The “off-gel” IPG—isoelectric focusing principle described by Faupel and al (GB0010957.9) consists in a device, method and kit for separating charged and neutral compounds and recovery of said compounds in a solution, particularly in an ampholyte-free or buffer-free solution, said device comprising: (a) a chamber, including: an inlet end with a means to introduce an analyte solution, an outlet end with a means to retrieve or recycle separated fractions, a front wall and a rear wall, and one wall of the chamber composed of the chemical buffering system (b) a means for producing an electrical current across said chemical buffering system whereby a potential difference is produced resulting in said charged and neutral compounds to be differentially separated, (c) a means for collecting separated fractions, particularly in solution, most particularly in an ampholyte-free or buffer-free solution and (d) optionally, to recycle separated fractions. The pH of this solution is controlled by placing it in a chamber with a chemical buffering system, for example, an immobiline™ gel, a fluid solidified in a polymer matrix, a fritted glass, a filter or any combination thereof. This chemical buffering system serves to fix the pH in its portion contacting the analyte solution, thereby allowing discrimination between ions and compounds that are globally neutral at this pH. An electric field is applied across said chemical buffering system, and the shape of the chamber is designed in such a manner that the electric field penetrates within this chamber, thereby generating a migration flux of the charged species present in solution. The separation induced by the migration of charged compounds directly occurs in the analyte solution.

One of the disadvantages of all of the above-described methods is that they often rely on the use of matrices made comprising acrylamide, a toxic molecule.

The present invention overcomes the drawbacks of the previous technologies. In other terms, the present invention provides compositions and membranes enabling electro-filtration methods which allow to filtrate, fractionate, concentrate, separate or fixate a desired molecule from a mixture of molecules characterized in that they have a high sample load, a high resolution, and extremely low loss of the molecules within the filter membranes, as well as easy recovery of the isolated molecules. Moreover, the compositions and membranes of the invention have the important advantage of being composed of non-toxic substances, being thus advantageously suitable for preparative processes.

This solution to the drawbacks of the previous technologies is based on the facts that the present inventors have surprisingly discovered that N-acryloyl-tris(hydroxymethyl)aminomethane (hereinafter “NAT”), a non-toxic molecule, can be covalently bound to a support, a glass support in particular, and that acrylamido buffers can be covalently linked to NAT (either free or covalently bound to a support).

The use of gels composed of NAT has already been suggested by Kozulic and Mosbach (WO-A-88/09981 and Analytical Biochemistry, 1987, 163:506-512) as a replacement for, or in combination with, acrylamide. The authors of these documents did however not realise that NAT could be covalently bound to a support, thus providing advantageous membranes. Moreover, although the above publications refers to isoelectric focusing, their authors used ampholytes, which are not covalently bound to the gel, and did not realise that acrylamido buffers can be covalently linked to NAT thus leading to advantageous compositions, gels and/or membranes for isoelectric focusing or filtration.

### SUMMARY OF THE INVENTION

The present inventors have surprisingly discovered that N-acryloyl-tris(hydroxymethyl)aminomethane (hereinafter “NAT”), a non-toxic molecule, can be covalently bound to a support, a glass support in particular.

The present inventors have moreover discovered that acrylamido buffers can be covalently linked to NAT (either free or covalently bound to a support).

The present invention thus relates to membranes comprising N-acryloyl-tris(hydroxymethyl)aminomethane (NAT) covalently linked to a support, in particular glass, glass fibres, fritted glass or fibreglass.

The membranes of the invention may further comprise an isoelectric buffer covalently bound to the NAT, for instance an acrylamido buffer or an Immobiline™. Such membranes are particularly well suited for isoelectric trapping processes and for the preparative filtration of molecules.

The present invention also encompasses compositions comprising an isoelectric buffer covalently bound to NAT, which compositions can be used to provide gels for isoelectric focusing analyses. Agarose can be included into said gels in order to improve their physical stability.

The present invention further encompasses devices comprising a membrane or composition of the invention. Said devices of the invention may also comprise two or more membranes of the invention, wherein a chamber or compartment is present between said membranes. When the devices of the invention comprise several chambers or compartments, the volume of said chambers or compartments can be either identical to one another or different from one another. Compartments or chambers having different volume allow to easily concentrate desired molecule or to dilute an undesired molecule.

The membranes, compositions or devices of the invention can be used for the separation or filtration of molecules, preferably electro-filtration. Said membranes, compositions or devices of the invention can also be used to concentrate, fractionate or fixate desired molecules.

Preferred uses of the membranes, compositions or devices of the invention are isoelectric filtration and/or isoelectric trapping.

The molecules filtrated and/or isolated according to the present invention can be charged molecules, preferably proteins, polypeptides, peptides, amino acids, nucleic acid molecules, polynucleotides, oligonucleotides, nucleotides, or homologues or analogues thereof, and/or combination thereof. Said molecules can be antibodies or fragments thereof, DNA, RNA, cDNA, mRNA, or PNA. In a preferred embodiments, said molecules are siRNA or miRNA molecules.

The present invention also encompasses the processes of preparing the membranes, compositions or devices of the invention.

### FIGURES

FIG. 1: Graph of correlation between NAT and acrylamide membranes

FIG. 2: Schematic representation of exemplary mold blocks

FIG. 3: Ampholine Gel 3.5-9-5: Antibody separated from crude serum. 98 represents the mixture before any separation. After separation, fractions (98+) and (98−) were collected. The pI of the antibody is 7.8, then during the separation this protein migrated below the 7.35 membrane whereas the serum was maintained above it. The dilution of serum is ½ in 98+ whereas it is 1/10 in 98

FIG. 4: Time needed to separate antibody from crude serum: A; After 1 hour of separation, B; After 2 hours of separation, C; After 3 hours of separation, D; After 4 hours of separation, E; After 5 hours of separation.

FIG. 5: Separation of Eglin C and βLactoglobulin. “Starting Material” represents the mixture before any separation. After separation, fractions +(78+ and 79+) and −(78− and 79−) were collected. During the separation Eglin migrated below the 5.45 and 5.55 membranes whereas the βLactoglobulin was maintained above it. The coloration of the Starting Material is more dark than the samples after separation. This is due to the fact that after adding the sample above the membrane, an additional volume must be considered: the volume below the membrane, which dilutes samples 78+/− and 79+/−.

FIG. 6: Schematic representation of exemplary multichambers device suitable for use with the membranes of the invention.

### DETAILED DESCRIPTION OF THE INVENTION

The present invention relates to the use of non-toxic molecule N-acryloyl-tris(hydroxymethyl)aminomethane (NAT) to prepare membranes or to prepare gels for use in an isoelectric focusing (IEF) process. In view of the fact that the membranes/gels of the prior art comprise acrylamide, which is toxic, and/or ampholytes which are not covalently bound, the present invention solves the technical problem of providing improved membranes/gels for the filtration/separation of molecules.

The membranes and gels according to the present invention allow for a rapid and convenient filtration, separation and/or purification of an amphoteric or neutral molecule or chemical compound, without the need of the toxic acrylamide (the use of which is not approved by the Food and Drug Administration). Furthermore, the membrane according to the invention allows rapid separation of a desired molecule from a mixture of other molecules by way of passage over a single membrane. In addition, the porosity of the membranes is easily adjusted by the choice of the support and/or by the pI thereof, which pI is determined on the choice of the acrylamido buffer used, in a fashion that is well known to the skilled person.

A permeable membrane can be made of a support which may be glass microfiber filters, such as Whatman GF/D filters to which NAT molecules is coupled. The process for preparing the membranes is based on the surprising abilities of NAT to covalently bind to a support and of acrylamido buffers to become covalently linked to NAT, for instance in a gel, thus fixing the buffering pH of the gel at any desired value. This overcomes the drawbacks of the previously used acrylamide, which is highly toxic, and also allows a quicker separation/filtration of molecules, especially in preparative processes since e.g. the FDA does not approve the use of acrylamide.

The molecule to be separated may be a protein, enzyme or smaller peptide having at least two amino acids or a compound containing a peptide- or protein moiety, e.g. a glycoprotein, but may also be a nucleic acid (single- or double-stranded), complex lipid or complex carbohydrate. Without wishing to be bound by theory, these molecules are amphoteric and can be kept in an isoelectric or uncharged state under the conditions of the purification process and at the time when the separation from the undesired accompanying chemical compound(s) actually takes place.

Contrary thereto, molecules that have an electrical charge under the conditions of the purification process and at the time when the separation from the desired chemical compound actually takes place will further migrate towards the electrodes, thus traversing the compartment/chamber wherein the desired molecule is retained. Another example of a undesired molecule which can be eliminated by this process is a salt, e.g. an alkali metal salt, for instance sodium chloride.

The filtration performed using the membrane according to the present invention may be performed using any solvent allowing for the necessary flow of compounds, e.g. water or a mixture of water with a suitable alcohol, e.g. a lower alkanol, for example methanol or ethanol, or an aqueous solution containing e.g. commonly used buffers, urea, detergents or any other water-miscible organic solvent. Buffers suitable for the filtration of the invention include, but are not limited to the buffers commonly used in the field, for instance phosphoric acids, sodium hydroxide or any composition of ampholytes.

In one embodiment, the membrane according to the present invention is placed between two chambers capable of holding a liquid and comprising electrodes. The membrane, being permeable, allows for passage of liquids and molecules between the chambers. In another aspect of the invention, two membranes according to the invention (with the same or different pH values) are placed on two sides of a chamber in order to perform a separation. This separation can take place in any of the known devices comprising two subcompartments that are separated from each other by septa-like structure, for example, monofilament screens, membranes, gels, filters, and fritted discs. Suitable devices are the multicompartments electrolyzers with isoelectric membranes which were introduced for processing large volumes and amounts of proteins to homogeneity (Righetti, P. G., Wenisch, E. and Faupel, M., 1989, J. Chromatogr. 475:293-309; Righetti, P. G., Wenisch, E., Jungbauer, A., Katinger, H. and Faupel. M., 1990, J. Chromatogr. 500:681-696; Righetti, P. G., Faupel, M. and Wenisch, E., 1992, In: Advances in Electrophoresis, Vol. 5, Chrambach, A., Dunn, M. J. and Radola, B. J., eds., VCH, Weinheim, pp. 159-200). This purification procedure, based on isoelectric focusing, progresses under recycling conditions, by keeping the protein macroions in a reservoir and continuously passing them in the electric field across a multicompartment electrolyzer equipped with zwitterionic membranes. In this system, the protein is always kept in a liquid vein (thus it is not lost by adsorption onto surfaces, as customary in chromatographic procedures) and it is trapped into a chamber delimited by two membranes having pIs encompassing the pI value of the protein being purified. Thus, by a continuous titration process, all other impurities, either non isoelectric or having different pI values, are forced to leave the chamber, in which the protein of interest will ultimately be present as the sole species, characterized by being isoelectric and isoionic as well (note that the isoelectric and isoionic points of a protein can differ to some extent in the presence of counterions). The devices can be used for separation and purification of proteins and peptides, i.e. for removing contaminants (including other macroions and salts) from the protein of interest (Faupel, D. M. and Righetti, P. G., U.S. Pat. No. 4,971,670, Nov. 20, 1990). Similar multiple subcompartments devices are described in U.S. Pat. No. 4,971,670 issued to Faupel et al., U.S. Pat. No. 5,173,164 issued to Egen et al., U.S. Pat. No. 4,963,236 issued to Rodkey et al., and U.S. Pat. No. 5,087,338 issued to Perry et al., all of which disclose devices comprising series of parallel spacers that are separated from each other by septa, which results in an essentially parallel array of subcompartments. A large number of other similar devices have been disclosed in patents and other publications. In all such devices, electrodes are provided at the ends of the assembly of subcompartments for the application of an electrical field.

The actual separation is performed by applying an electric field across the membrane, by placing the cathode in one chamber and the anode in the other chamber. The electric field is generated by the power supply. Any voltage the system can tolerate may be used, e.g. 100 to 10000 volt, especially 500 to 10000 volt, preferably 500 to 5000 volt, e.g. 500, 1000, 5000 or even 10000 volt, provided the generated heat can be dissipated by proper cooling. At equilibrium, typical values are e.g. 1000 volt, 3 mA and 3 W or 500 volt, 10 mA and 5 W.

Amphoteric isoelectric immobilized pH-membranes do not comprise a pH-interval but have throughout the membrane the same pH-value. The manufacture of such membranes is routine for the skilled person and is similar to the manufacture of matrices (such as gels comprising acrylamide) with pH-gradients. No gradient mixer is required and no glycerol is necessary for preparing a density gradient of the invention.

Previously described membranes useful for IEF are manufactured by polymerization, preferably around neutral pH, at 50° C. in a forced-ventilation oven for 1 hour, of a solution of monomers (in general 10-15% acrylamide and 3-4% crosslinking agent: N,N′-methylene-bis- acrylamide) containing variable amounts of buffering and titrant isoelectric buffer (Immobilines™), Ampholines™ in the ratios needed to generate the desired isoelectric point together with suitable polymerisation catalysts and water.

It is important that the membranes have a good buffering capacity at their isoelectric point in order to prevent electroendosmosis, a term denoting bulk liquid flow through the membrane caused by the presence or acquisition of a net electrical charge. However, the Immobiline™ molarity should preferably not exceed 50 mM of each Immobiline™ in the membrane.

Ampholines™ are low-molecular-weight amphoteric substances, i.e. ampholytes, which contrary to Immobilines™, are not fixed to the acrylamide/N,N′-methylene-bis-acrylamide or NAT polymer and are therefore able to contribute to the electrical conductivity. Mixtures of many amphoteric substances such as amino acids and peptides and some amphoteric and non-amphoteric buffer components can act as suitable ampholytes. However, the great majority of iso-electric focusing experiments are performed with the aid of commercial ampholyte mixtures. The most widely used of these, is marketed by LKB Produkter AB under the brand name Ampholines™. They consist of synthetic mixtures of polyaminopolycarboxylic acids with molecular weights mostly in the region of 300-600. Other products can be used which contain sulphonic or phosphonic acid groupings in addition to the amino and carboxylic acid groups. These products (Servalyts™, Serva-Feinbiochemica GmbH; Biolytes™, Bio-Rad Laboratories; Pharmalytes™, Pharmacia AB) have recently been compared with the Ampholines™ and shown to have a similar performance.

The amidoacrylo buffers Immobilines™ are acrylamide derivatives with the general structure CH₂═CH—CO—NH—R where R contains either a carboxylic acid or a tertiary amino group. Immobilines™ are designed for co-polymerization with acrylamide and N,N′-methylene-bis-acrylamide in order to produce immobilized pH-gradients. Each derivative has a defined and know pK-value. N-(3- Dimethylamino-propyl)-methacrylamide having a pK-value of 9.5 may be mentioned as an example of a methacrylamide derivative being analogous to an Immobiline™.

According to the prior art, acrylamide may be replaced by e.g. methacrylamide, and N,N′-methylene-bis-acrylamide may be replaced by any other suitable crosslinker, e.g. other suitable acrylamide derivatives. Those proposed replacement do however not circumvent all of the drawbacks known in the art. The inventors have however surprisingly discovered that the toxic acrylamide could be advantageously replaced by the non-toxic NAT.

After co-polymerisation the Immobilines™ are covalently bound, i.e. immobilized, and do not contribute anything to the conductivity of the pH-gradient or pH-membrane. However, the Immobilines contribute to the buffering and titrant capacity.

Preferably, the pH-membranes are cast somewhere within a pH-range from about 3 to about 10, depending on the Immobilines™ and Ampholines™ available. If the compound of interest is amphoteric, the pH-values in the two membranes facing the flow chamber have to be set just above and below or equal to the isoelectric point of said amphoteric substance with the precision required to keep it in the isoelectric state all the time.

In this modified isoelectric focusing technique, the protein of interest is not driven electrophoretically into the membrane (from which it would have to be recovered by an additional purification step), but is kept in a charged state in the liquid from which it can be easily separated. As soon as the desired molecule enters the pH-fixed membrane which has be chosen for its ability to isolate said desired molecule, it loses its charge and reenters the liquid. This molecule will therefore not pass through said membrane and will remain in the chamber/compartment in front of said membrane. This way, at a set pH, only molecules which are charged at that pH, e.g. the undesired molecules will be able to pass through the membrane.

With pH-membranes, it is in most cases possible to set the conditions so that the pH of the membrane is just below the isoelectric point (pI) of the component of interest. If needed, in a second separation step, the pH of the membrane may be set to a value just above the pI of the desired compound. (Of course, the manufacture of suitable immobilized pH gradients (IPGs) may be difficult in the comparatively rare cases where the desired substance has an extremely high or low pI.) The desired molecule, having a discrete isoelectric point, will thus be isoelectric only in the narrow pH gap delimited by the two immobilized pH-gradients or pH-membranes chosen.

If the compound of interest is amphoteric, this gap comprises normally 0.05 to 0.2 pH-units; however, gaps comprising down to 0.001 pH-units can be also achieved. It is also possible that the gap comprises 0 pH-units, i.e. the pH-values in the extremities correspond to the isoelectric point of the desired compound. This means that there is no pH-gap at all, but only a fluid gap between two gel phases.

The skilled person will readily understand that if the compound of interest is neutral, the pH-values in the membranes are not chosen in respect to the desired compound, but in respect to the undesired amphoteric or charged compounds, in the sense that said undesired compounds will be trapped within dicrete pH-gaps. The neutral compound will never enter the pH-gradients, irrespective of the boundary conditions in the membranes facing the flow chamber. In addition to its precision in setting the boundary conditions, the unlimited stability of IPGs with time will automatically ensured that the pH gradient never drifts so that the isoelectric conditions for the compound under purification will be constantly found in the hydraulic flow, especially in the flow-chamber, and not elsewhere, e.g. within the anodic or cathodic gel phases.

The process according to the present invention has at least the following major advantages:
(a) non-toxicity of the membranes;
(b) extremely high sample recoveries, approaching 100%, as the desired compound (e.g. the protein) under purification never enters the gel phase, but is kept uncharged, e.g. in an isoelectric state, during the entire purification step in the liquid phase;
(c) large sample loads, as the compound to be purified, e.g. the protein feed, may be kept circulating between a separate reservoir and a flow chamber and only small amounts need be present at any given time in the electric field;
(d) a high resolving power, depending on the narrowness the pH interval selected across the isoelectric point (pI) of the desired compound, e.g. protein;
(e) automatic removal of any salts or buffers accompanying the compound (e.g. the peptide or protein) of interest, which means that the present process can also be used for electrodialysis (desalting process). Especially the removal of monovalent ions of strong acids or bases, e.g. the acids HCl, HBr, HI, HNO₃, HClO₄, H₂SO₄, or the bases LiOH, NaOH, KOH, RbOH, CsOH, Ca(OH)₂, Sr(OH)₂, Ba(OH)₂, is very easy. For the removal of monovalent ions of weak acids and bases, e.g. ammonium and acetate, it is advantageous to use the amphoteric isoelectric Immobiline™ membranes described below or rather short pH-gradients, i.e. gradients comprising only a comparatively small pH-range, e.g. 0.5 to 1.0 pH-units, substantially removed from the pK-values of the respective weak acids and bases. The removal of multivalent ions, e.g. sulphate, phosphate and citrate, takes more time, possibly due to the interaction of these species with the Immobiline™ matrix, and is best carried out under outside pH-control, e.g. with a pH-stat, since the faster removal of the monovalent counterion can cause the solution in chamber to become acidic or alkaline. Rapid desalting of protein samples for a variety of uses, e.g. enzyme reactions or ligand binding studies, is one of the problems currently faced in biochemistry. Any salt content in the sample feed (already at 1 mM concentration) inhibits the transport of non-isoelectric proteins, perhaps because of the much larger current fraction carried by the ions themselves as opposed to proteins. In addition, high salt levels in the sample reservoir may form cathodic and anodic ion boundaries, alkaline and acidic, respectively, which may hamper protein migration and even induce denaturation. In segmented (as well as in conventional) IPG gels, practically any level of salt present in the sample zone inhibits its electrophoretic transport. Therefore, the best way to efficiently eliminate protein impurities from an isoelectric component is to introduce an already desalted protein feed into the segmented IPG apparatus. However, elimination of protein impurities can be achieved, although at a slower rate, even in the presence of salts in the sample. In the latter case, salt levels should be kept at the minimum compatible with protein solubility (e. g. 5 mM or lower) and external pH control should be exerted (e.g. with a pH-stat) so as to prevent drastic pH changes in the sample feed, brought about by the generation of boundaries produced by the salt constituents. In quite a few cases, a minimum salt concentration might be needed in the sample phase during the electrophoresis for preventing protein aggregation and precipitation due to too low an ionic strength at or in the vicinity of the isoelectric point. For that purpose, an external hydraulic flow may be used, replenishing the salt loss due to combined electric and diffusional mass transports (similar to the concept of Rilbes' steady-state rheoelectrolysis, H. Rilbe, J. Chromatography 159, 193-205 [1978]).

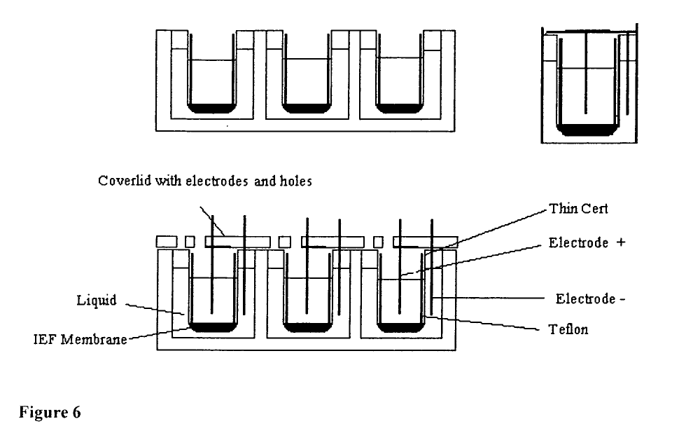

An exemplary multichambers device is depicted in FIG. 6 and consists of a box, electrodes, and a part between the electrodes denominated “membrane unit”. The coverlid of the box has holes permitting extraction, introduction or handling of solutions. Electrodes are fixed as depicted in FIG. 6. The device comprises a unit of two compartments divided by a permeable membrane and a coverlid with fixed electrodes. An electrode is placed in each of the compartments and the liquid to be processed is contained in or allowed to flow through the permeable membrane of the invention. When one electrode is made positive and the other negative the ions of any ionisable compounds contained in the liquid will migrate through the permeable membrane. The positively charged ions towards the negative electrode and the negatively charged ions towards the positive electrode. The membranes of the invention and the membrane unit are preferably placed as depicted in FIG. 6. Once an electric field is applied to the pH buffered membranes, they become “pI selective” and will only allow amphoteric species such as e.g. proteins or peptides to move towards the opposite electrode.

The shape of the chamber can be designed in such a manner that the electric field will penetrate within it and thereby generates a migration flux of the charged species present in the solution so that the desired molecules proteins are always kept in a liquid vein.

The present invention also allows to run in “electro filtration modes”, proteins mixtures, cells extracts, peptides and other samples or salts in devices of the invention. To apply such devices to electrophoresis, electrodes compartments are included.

In one embodiment, filtration membranes are housed in the “membrane unit” providing a modular system which has substantial advantages of the known prior art. The used membrane unit is easily removed and a new membrane unit may be simply inserted. It should be noted that the different possible designs are applicable to other applications like electro dialysis, ELISA and reverse osmosis devices and combinations of applications.

The following examples illustrate the invention without limiting it in any way.

### EXAMPLES

### Example 1

### Preparation of Membranes

The choice of the compositions of NAT (N-acryloyl-tris(hydroxymethy)aminomethane; available from FLUKA, Buchs, Switzerland, or from Elchrom Scientific AG, Cham, Switzerland), N, N′-Methylenebisacrylamide, and acrylamido buffers concentrations and ratio will vary depending on the size of the proteins to be separated. Selection of an appropriate gel composition is a key consideration for membranes. Two factors are particularly important: the gel porosity and the concentration of acrylamido buffers in the gel. Both properties are determined by the relative amounts of NAT and cross-linker which are usually expressed by the relationship:

$$T = \frac{g NAT + cross - linker}{100 ml of solution}$$

$$C = \frac{g cross - linker}{\% T}$$

To establish new protocols allowing the fabrication of membranes; protocols based on acrylamide monomer were first used using, published by Amersham: (80-6350-18 Rev.A/2-97). Table 1 show an example acrylamido buffers mixtures to be used to produce isoelectric membranes. We have established a correlation graphic using NAT (FIG. 1) as compared to acrylamide.

TABLE 1
Acrylamido buffers mixtures for isoelectric membranes

| | | | Immobilino pK buffers used (μl) | | | | | | 1M Tris | 1M Acetic |
|---|---|---|---|---|---|---|---|---|---|---|
| | | pH | 3.6 | 4.6 | 6.2 | 7.0 | 8.5 | 9.3 | base (μl) | acid (μl) |
| Range | 1 | 4.00 | 401 | 71 | 311 | 0 | 0 | 0 | 79 | 0 |
| pH | 2 | 4.05 | 392 | 86 | 304 | 6 | 0 | 18 | 77 | 0 |
| 4.00-7.00 | 3 | 4.10 | 388 | 94 | 300 | 9 | 0 | 27 | 76 | 0 |
| | 4 | 4.15 | 383 | 101 | 296 | 12 | 0 | 36 | 74 | 0 |
| | 5 | 4.20 | 379 | 109 | 293 | 15 | 0 | 45 | 73 | 0 |
| | 6 | 4.25 | 375 | 116 | 289 | 18 | 0 | 54 | 71 | 0 |
| | 7 | 4.30 | 371 | 124 | 286 | 22 | 0 | 63 | 70 | 0 |
| | 8 | 4.35 | 366 | 131 | 282 | 25 | 0 | 72 | 68 | 0 |
| | 9 | 4.40 | 362 | 139 | 278 | 28 | 0 | 81 | 66 | 0 |
| | 10 | 4.45 | 358 | 146 | 275 | 31 | 0 | 90 | 65 | 0 |
| | 11 | 4.50 | 353 | 154 | 271 | 34 | 0 | 99 | 63 | 0 |
| | 12 | 4.55 | 349 | 161 | 267 | 37 | 0 | 108 | 62 | 0 |
| | 13 | 4.60 | 345 | 169 | 264 | 40 | 0 | 118 | 60 | 0 |
| | 14 | 4.65 | 340 | 177 | 260 | 43 | 0 | 127 | 58 | 0 |
| | 15 | 4.70 | 336 | 184 | 257 | 46 | 0 | 136 | 57 | 0 |
| | 16 | 4.75 | 332 | 192 | 253 | 49 | 0 | 145 | 55 | 0 |
| | 17 | 4.80 | 328 | 199 | 249 | 52 | 0 | 154 | 54 | 0 |
| | 18 | 4.85 | 323 | 207 | 246 | 55 | 0 | 163 | 52 | 0 |
| | 19 | 4.90 | 319 | 214 | 242 | 59 | 0 | 172 | 51 | 0 |
| | 20 | 4.95 | 316 | 222 | 239 | 62 | 0 | 181 | 49 | 0 |
| | 21 | 5.00 | 310 | 229 | 235 | 65 | 0 | 190 | 47 | 0 |
| | 22 | 5.05 | 306 | 237 | 231 | 68 | 0 | 199 | 46 | 0 |
| | 23 | 5.10 | 302 | 245 | 228 | 71 | 0 | 206 | 44 | 0 |
| | 24 | 5.15 | 298 | 252 | 224 | 74 | 0 | 217 | 43 | 0 |
| | 25 | 5.20 | 293 | 260 | 220 | 77 | 0 | 226 | 41 | 0 |
| | 26 | 5.25 | 289 | 267 | 217 | 80 | 0 | 235 | 40 | 0 |
| | 27 | 5.30 | 285 | 275 | 213 | 83 | 0 | 244 | 38 | 0 |
| | 28 | 5.35 | 280 | 282 | 210 | 86 | 0 | 253 | 36 | 0 |
| | 29 | 5.40 | 276 | 290 | 206 | 89 | 0 | 262 | 35 | 0 |
| | 30 | 5.45 | 272 | 297 | 202 | 92 | 0 | 271 | 33 | 0 |
| | 31 | 5.50 | 268 | 305 | 199 | 95 | 0 | 280 | 32 | 0 |
| | 32 | 5.55 | 263 | 313 | 195 | 99 | 0 | 289 | 30 | 0 |
| | 33 | 5.60 | 259 | 320 | 192 | 102 | 0 | 298 | 28 | 0 |
| | 34 | 5.65 | 255 | 328 | 188 | 105 | 0 | 307 | 27 | 0 |
| | 35 | 5.70 | 250 | 335 | 184 | 108 | 0 | 316 | 25 | 0 |
| | 36 | 5.75 | 246 | 343 | 181 | 111 | 0 | 325 | 24 | 0 |
| | 37 | 5.80 | 242 | 350 | 177 | 114 | 0 | 334 | 22 | 0 |
| | 38 | 5.85 | 237 | 358 | 173 | 117 | 0 | 343 | 21 | 0 |
| | 39 | 5.90 | 233 | 365 | 170 | 120 | 0 | 353 | 19 | 0 |
| | 40 | 5.95 | 229 | 373 | 166 | 123 | 0 | 362 | 17 | 0 |
| | 41 | 6.00 | 225 | 381 | 163 | 126 | 0 | 371 | 16 | 0 |
| | 42 | 6.05 | 220 | 388 | 159 | 129 | 0 | 380 | 14 | 0 |
| | 43 | 6.10 | 216 | 396 | 155 | 132 | 0 | 389 | 13 | 0 |
| | 44 | 6.15 | 212 | 403 | 152 | 136 | 0 | 398 | 11 | 0 |
| | 45 | 6.20 | 207 | 411 | 148 | 139 | 0 | 407 | 9 | 0 |
| | 46 | 6.25 | 203 | 418 | 145 | 142 | 0 | 416 | 8 | 0 |
| | 47 | 6.30 | 199 | 426 | 141 | 145 | 0 | 425 | 6 | 0 |
| | 48 | 6.35 | 195 | 433 | 137 | 148 | 0 | 434 | 5 | 0 |
| | 49 | 6.40 | 190 | 441 | 134 | 151 | 0 | 443 | 3 | 0 |
| | 50 | 6.45 | 186 | 448 | 130 | 154 | 0 | 452 | 2 | 0 |
| | 51 | 6.50 | 182 | 456 | 126 | 157 | 0 | 461 | 0 | 0 |
| | 52 | 6.55 | 177 | 464 | 123 | 160 | 0 | 470 | 0 | 1 |
| | 53 | 6.60 | 173 | 471 | 119 | 163 | 0 | 479 | 0 | 2 |
| | 54 | 6.65 | 169 | 479 | 116 | 166 | 0 | 488 | 0 | 3 |
| | 55 | 6.70 | 165 | 486 | 112 | 169 | 0 | 497 | 0 | 4 |
| | 56 | 6.75 | 160 | 494 | 108 | 172 | 0 | 506 | 0 | 4 |
| | 57 | 6.80 | 156 | 501 | 105 | 176 | 0 | 515 | 0 | 5 |
| | 58 | 6.85 | 152 | 509 | 101 | 179 | 0 | 524 | 0 | 6 |
| | 59 | 6.90 | 147 | 516 | 98 | 182 | 0 | 533 | 0 | 7 |
| | 60 | 6.95 | 143 | 524 | 94 | 185 | 0 | 542 | 0 | 8 |
| | 61 | 7.00 | 139 | 532 | 90 | 188 | 0 | 551 | 0 | 9 |

For the present example, the following proportions have been chosen for a stock solution of NAT at 17.5%: 5.64 g of N-acryloyl-tris(hydroxymethy)aminomethane (NAT); 0.16 g of N, N′-Methylenebisacrylamide; and 26.5 g of water.

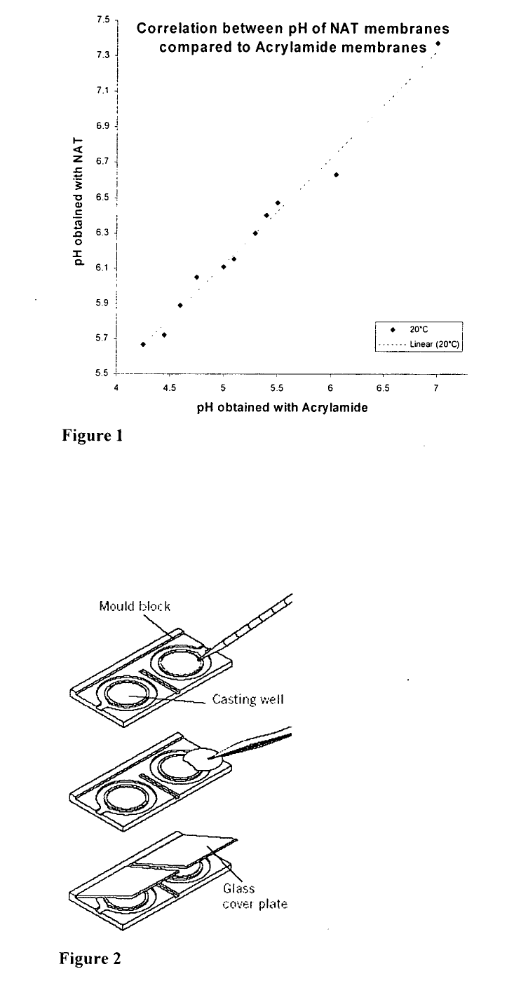

Said stock solution has been obtained according to the following protocol:
Deionize, filter and degas the NAT/bisacrylamide monomer stock solution.
Remove monomer buffers from 4° C. storage and allow to warm to room temperature for 30 minutes.
Coat clean dry cover plates for each mold block (FIG. 2) with 1 ml of Repel-Silane™ solution and allow to dry several minutes. Repel-Silane™ prevents membranes adhesion to the cover plates.
Prepare 1 ml of 40% ammonium persulfate (APS) immediately before use (0.4 g APS in 1 ml ddwater) and TEMED (,N,N′,N′-tetramethylethylenediamine; 99.9%).

Preparation of the isoelectric membrane:

The pH of each membrane set is selected so that the different molecules in the solution (whose different pI values are known) are trapped in separate chambers.

Prepare, deionize, filter and degas enough NAT/bisacrylamide monomer stock solution

Remove monomer buffers from 4° C. storage and allow to warm to room temperature for 30 minutes

Coat the clean, dry, cover plates for each mold block (FIG. 2) with 1 ml of Repel-Silane™ solution and allow to dry several minutes. Repel-Silane™ prevents membranes adhesion to the cover plates. Wash extensively the cover plates.

Follow the acrylamido buffer protocol listed in Table1 to create the isoelectric membranes. Be sure to measure the monomer buffer solution pH before adding the NAT/bisacrylamide stock solution.

Prepare 1 ml of 40% ammonium persulfate (APS) immediately before use (0.4 g APS in 1 ml ddwater). Add NAT/bisacrylamide stock solution, 5 μl of TEMED (99.9%), and 10 μl of 40% APS to the monomer buffer solution and mix to obtain the “gel solution”.

Deliver 3 ml of the “gel solution” into each of the 2 wells in the mould block

A Whatman GF/D, 4.7 cm filter is wetted by placing it at an angle into a well and allowing capillary action to saturate it with the solution. Once saturated, lower the filter into its well, gently pressing it into place with gloved fingers.

Pipette an additional 2 ml of the “gel solution” onto each filter.

Lower the glass cover plate, with the silane-coated side toward the membrane, onto the membrane, and allow excess gel solution to escape from between the plate and the acrylic block.

Take care to avoid trapping air under the cover plate. The reason therefore is that air retards polymerization and that air bubbles may cause holes in the membrane.

Incubate the membranes at 50° C. for 1 hour.

### Example 2

### Experimental Setup

A 8 μm porosity Thin Cert™ (sealed PET capillary pore membrane, with hanging geometry and made of transparent polystyrene inserts for 6-, 12- and 24-well multiwell plates with 0.4 μm, 1.0 μM, 3.0 μm and 8.0 μm pore sizes, obtainable form Greiner Bio-One, Frickenhausen, Germany) was placed in an apparatus according to FIG. 6. This apparatus is composed of a coverlid with electrodes, as depicted in FIG. 6. The 8 μm porosity Thin Cert™ has been placed in the apparatus as depicted in FIG. 6. Thereafter, the isoelectric membrane was placed therein and kept in place by a Teflon® ring. To avoid air bubbles being trapped in its holes, the Thin Cert™ was agitated 30 minutes in water before use. The separating fluids were introduced into each part of the apparatus, the sample to be analyzed was then introduced on the anode side and voltage applied. 400 μl buffer was placed above the membrane (anodic side) and 500 μl thereof under the membrane (cathodic side). At the end of the experiment, fractions were collected and analyzed.

### Example 3

### Preparative Separation of an Antibody with an Isoelectric Point of pI 7.8. from Crude Serum.

To 100 μl of 1/1 diluted serum was added 300 μl H₃PO₄ 10 mM and 3 μl antibody at 54.7 mg/ml. This solution was placed on the anodic side of the membrane. On the cathodic side of the membrane, 500 μl H₂O, together with 0.0001% Pharmalyte 8-10 (GE Healthcare Bio-science AB, Uppsala, Sweden) was placed. A voltage of 100V, current of 2 mA, and power of 2 W was applied for 30 minutes followed by a second step voltage of 200V, current of 2 mA and power of 2 W applied for 2 hours and 30 minutes.

### Results of the Experiment

The analysis was conducted on a Ampholine™ Gel 3.5-9.5 plate (GE Healthcare Bio-science AB, Uppsala, Sweden). The lower limit for sample concentration depends both on the volume of the sample applied and on the sensitivity of the detection method used to develop the gel. A sample volume of 80 μl was applied. The following conditions were used:

500V—25 mA—5 W during 40 minutes

1000V—25 mA—5 W during 40 minutes

1500V—25 mA—5 W during 1 h 30 minutes

Fixing and Staining conditions of the Ampholine™ Gel 3.5-9.5:

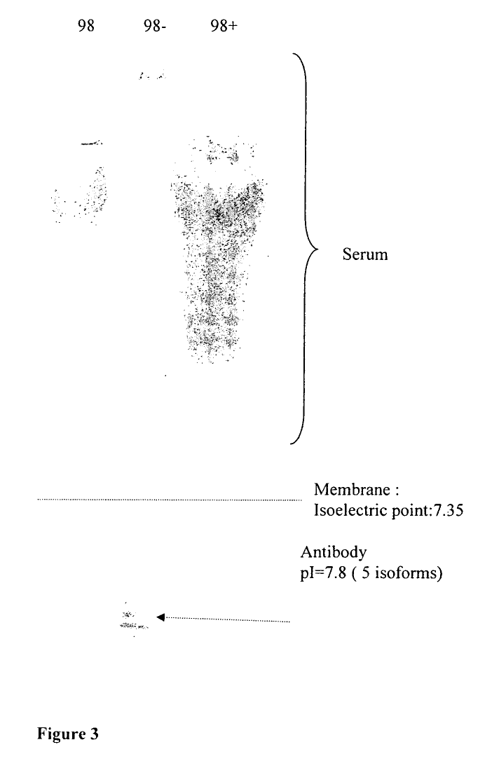

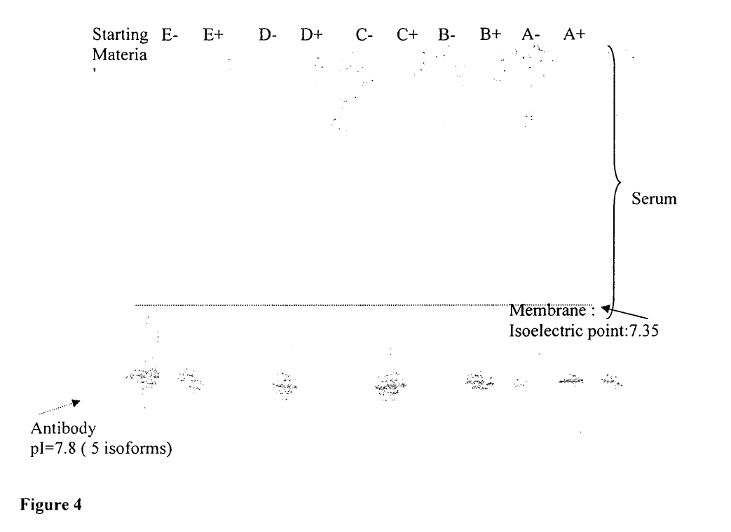

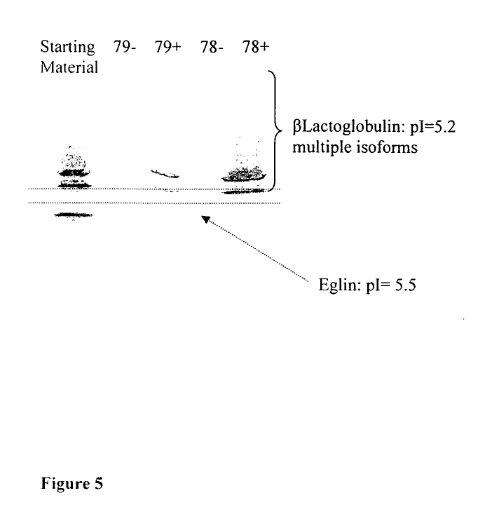

The following solutions were prepared: TCA 15%, Colloidal Coomassie Blue. The gel was fixed for 30 minutes in 100 ml 15% TCA, rinsed 2×5 minutes in distilled H2O, stained in 100 ml Coomassie Blue, then rinsed in distilled H2O. Results are shown in FIG. 3. These results show that this method allows to easily separate a protein for analytical characterizations from the disturbing crude serum.

### Example 4

### Eglin C (pI=5.55) and β Lactoglobulin Isoforms (pI=5.2. 5.3) to be Preparatively Separated with a pH 5.45 Isoelectric Membrane and in an Other Experiment a pH 5.55 Isoelectric Membrane

To 600 μl of protein mixture was added 100 μl 10 mM phosphoric acid. This solution was put on the anodic side of the membrane. On the cathodic side of the membrane, 1500 μl H₂O, together with 0.001% Pharmalyte 3-10 was placed. A voltage of 500V, current of 2 mA, and power of 2 W was applied for 4 hours. This setup was used as above, but now using membranes with a pH of 5.45 or pH 5.5, exp numbered 78 or 79, respectively.

### Analysis of the Experiment

An Immobiline™ Gel pH: 4-7 support was used (Immobiline™ DryPlate: GE Healthcare Bio-science AB, Uppsala, Sweden). A half of the gel was rehydrated in 39 ml H₂O+1 ml Pharmalyte™ 3-10 during 2 hours. The rehydrated gel was equilibrated at 1000V, 3 mA, 5 W during 1 hour before adding samples. Electrodes strips were soaked in H₂O+0.001% Pharmalyte™ 3-10.

The following conditions were used:

1000V—3 mA—5 W during 20 minutes

2500V—3 mA—5 W during 45 minutes

3500V—3 mA—5 W during 30 minutes

The gel (FIG. 5) was fixed and stained as in Example 4 above. The “Starting Material” represents the mixture before any separation. After separation, fractions from the anodic side (78+ and 79+) and from the cathodic side (78− and 79−) were collected. During the separation Eglin C migrated below the 5.45 and 5.5 membranes whereas the βLactoglobulin was maintained above it. The coloration of the Starting Material is darker than the samples after separation. This is due to the fact that after adding the sample above the membrane, an additional volume must be considered: the volume below the membrane, which dilutes samples 78+/− and 79+/−. The results show that the Eglin C (pI: 5.55) can be easily separated from the β lactoglobulin isoforms (pI's: 5.3 and 5.2) using a membrane of the invention.

1. A composition comprising a membrane, said membrane comprising N-acryloyl-tris(hydroxymethyl)aminomethane (NAT) covalently linked to a support.

2. The composition of claim 1 wherein the support is glass, including glass selected from glass fibers, fritted glass or fiberglass.

3. The composition of claim 1 further comprising an isoelectric buffer covalently bound to the NAT.

4. The composition of claim 3 wherein said isoelectric buffer is an acrylamido buffer.

5. A composition comprising an isoelectric buffer covalently bound to N-acryloyl-tris(hydroxymethyl)aminomethane (NAT).

6. The composition of claim 5 wherein said isoelectric buffer is an acrylamido buffer.

7. The composition of claim 5 wherein said composition is in the form of a gel.

8. The composition of claim 5 further comprising agarose.

9. A device comprising a N-acryloyl-tris(hydroxymethyl)aminomethane (NAT) membrane, wherein the membrane is optionally covalently linked to a support, optionally comprises the isoelectric buffer of claim 5 or both.

10. A device according to claim 9 comprising two or more NAT membranes, wherein a chamber or compartment is present between said membranes.

11. The device of claim 10 comprising several chambers or compartments, wherein the volume of said chambers or compartments is identical to one another or different from one another.

12. Use of a composition comprising N-acryloyl-tris(hydroxymethyl)aminomethane (NAT) membrane for the separation or filtration of molecules.

13. The use of claim 12 wherein said separation is electro-filtration, including filtration or isoelectric trapping.

14. Use of a composition of claim 5 for isoelectric focusing.

15. The use according to claim 12 wherein the molecule is a charged molecule, preferably selected from the group of proteins, polypeptides, peptides, amino acids, nucleic acid molecules, polynucleotides, oligonucleotides, nucleotides, and analogues thereof.

16. The use according to claim 12 wherein the molecule is a protein, preferably and antibody or a fragment thereof.

17. The use according to claim 12 wherein the molecule is a nucleic acid molecule, preferably a DNA, RNA, cDNA, mRNA, or PNA, more preferably a siRNA or a miRNA.

18. Use of a device according to claim 11 to concentrate a molecule.

19. A process for preparing a membrane of claim 1, the process comprising covalently linking N-acryloyl-tris(hydroxymethyl)aminomethane (NAT) to a support.

20. A process for preparing a composition according to claim 5, wherein the process comprises covalently binding an isoelectric buffer to N-acryloyl-tris(hydroxymethyl)aminomethane (NAT).
